(19) United States
(12) Patent Application Publication
(10) Pub. No.: US 2018/0036058 A1
(43) Pub. Date: Feb. 8, 2018

(54) ROTATIONAL CRYOGEN DELIVERY DEVICE

(71) Applicant: CSA Medical, Inc., Lexington, MA (US)

(72) Inventors: Wei Li Fan, Boston, MA (US); Brian M. Hanley, Reading, MA (US)

(21) Appl. No.: 15/668,004

(22) Filed: Aug. 3, 2017

Related U.S. Application Data
(60) Provisional application No. 62/370,975, filed on Aug. 4, 2016.

(51) Int. Cl. A61B 18/02

(52) U.S. Cl. CPC A61B 18/0218; A61B 2018/00488

(57) ABSTRACT

Approaches herein provide a cryogen delivery device including an opening through only a portion of a sidewall thereof, and a connector assembly which allows rotation of the opening for more controlled release of the cryogen from the device. In one approach, a connector couples an outer shaft to an inner shaft, the connector including a rotary component connected to a proximal end of the outer shaft to allow rotation of the outer shaft relative to the inner shaft. The opening extends only partially along a circumference of the outer shaft to provide a more controlled release of the cryogen from the delivery device.

### PRIORITY

This application claims the benefit of priority under 35 U.S.C. §119 to U.S. Provisional Patent Application Ser. No. 62/370,975, filed Aug. 4, 2016, which is incorporated by reference in its entirety and for all purposes.

### BACKGROUND OF THE DISCLOSURE

### Field of the Disclosure

The present disclosure relates generally to cryospray systems, cryogenic spray ablation and cryosurgery systems, and more particularly to a rotational cryogen delivery device for use in cryospray and cryosurgery systems.

### Discussion of Related Art

Cryospray treatment devices such as catheters are used for treatment of organic tissue. For example, cryogen is used for tissue ablation, which includes the removal or destruction of tissue, or of tissue functions. Traditionally, invasive and non-invasive surgical procedures are used to perform tissue ablation. These surgical procedures required the cutting and/or destruction of tissue positioned between the exterior of the body and the site where the ablation treatment is conducted, often referred to as the treatment site. Cryo ablation is an alternative in which tissue ablation is conducted by freezing diseased, damaged or otherwise unwanted tissue (collectively referred to herein as “target tissue”). Appropriate target tissue may include, for example, cancerous or precancerous lesions, tumors (malignant or benign), damaged epithelium, fibroses and any other healthy or diseased tissue for which cryo ablation is desired.

Cryo ablation may be performed using a system that sprays low pressure cryogen on the target tissue. Such systems are often referred to as cryospray systems, cryosurgery spray systems, cryosurgery systems, cryogen spray ablation systems or simply cryospray ablation systems. As used typically, cryogen refers to any fluid (e.g., gas, liquefied gas or other fluid known to one of ordinary skill in the art) that has a sufficiently low boiling point to allow for therapeutically effective cryotherapy and is otherwise suitable for cryogenic surgical procedures. For example, acceptable fluids may have a boiling point below approximately negative (−) 150° C. The cryogen may be liquefied nitrogen, as it is readily available. Other fluids such as argon and air may also be used. Additionally, liquid helium, liquid oxygen, liquid nitrous oxide and other cryogens can also be used.

During operation of a cryosurgery system, a user (e.g., clinician, physician, surgeon, technician, or other operator) sprays cryogen on the target tissue via a delivery catheter. The spray of cryogen causes the target tissue to freeze or “cyrofrost.” The user may target the cryospray visually utilizing endoscopy, bronchoscopy, pleuroscopy, or any other imaging assisted device or scope. The temperature range may be from negative 0° C. to (−)195° C., the latter temperature being the case for liquid nitrogen at low pressure.

One particular treatment technique uses direct spray or radial spray catheters. Direct spray requires angling of the bronchoscope during use to treat lesions. However, it is difficult to obtain the correct angle and/or predict accurate location of the spray. Furthermore, radial spray offers 360 degree spray, which disadvantageously may impact healthy tissue as well.

### SUMMARY OF THE DISCLOSURE

The present disclosure in its various approaches includes cryogen delivery apparatuses, system and treatment methods. Converted cryogen gas, such as nitrogen gas, may be released annularly (i.e., in a 360 degree fashion) within a body lumen. Should an area for treatment only be on a portion of a wall of a body lumen, healthy tissue may be treated with the cryogen unnecessarily. A rotational delivery device may allow for directed release of cryogen and thus directed treatment of desired tissue regions.

Approaches herein provide a cryogen device including an opening through only a portion of a sidewall thereof, and a connector assembly which allows rotation of the opening for more controlled release of the cryogen from the device. In one approach, a connector couples an outer shaft to an inner shaft, the connector including a rotary component connected to a proximal end of the outer shaft to allow rotation of the outer shaft relative to the inner shaft. The opening extends only partially along a circumference of the outer shaft to provide a more controlled release of the cryogen from the device.

In one approach, a rotational cryogen delivery device includes an outer shaft having a proximal end and a distal end, and an inner shaft having a proximal end and a distal end. The device further includes a connector coupling the outer shaft to the inner shaft. The connector may include a rotary component connected to the proximal end of the outer shaft to allow rotation of the outer shaft relative to the inner shaft, and an opening provided through a sidewall of the distal end of the outer shaft for delivery of a cryogen, wherein the opening extends only partially along a circumference of the outer shaft.

In another approach, a catheter includes an outer shaft having a proximal end and a distal end, an inner shaft having a proximal end and a distal end, and a connector coupling the outer shaft to the inner shaft. The connector may include a rotary component connected to the proximal end of the outer shaft to allow rotation of the outer shaft relative to the inner shaft, and an opening provided through a sidewall of the distal end of the outer shaft for delivery of a cryogen, wherein the opening extends only partially along a circumference of the outer shaft.

In another approach, a cryosurgery system includes an outer shaft having a proximal end and a distal end, an inner shaft having a proximal end and a distal end, and a connector coupling the outer shaft to the inner shaft. The connector may include a rotary component affixed to the proximal end of the outer shaft to allow rotation of the outer shaft relative to the inner shaft, and a stationary component affixed to the inner shaft, the rotary component rotatably coupled to the stationary component, and an opening provided through a sidewall of the distal end of the outer shaft for delivery of a cryogen, wherein the opening extends only partially along a circumference of the outer shaft.

In the above approach, and various other approaches according to the present disclosure, the connector of the rotational delivery device may include a stationary component affixed to the second shaft. The rotary component may be rotatably coupled with the stationary component. The second shaft may extend within an interior bore of the first shaft. An opening may be provided through the second shaft for delivery of the cryogen to the opening of the first shaft. The opening of the second shaft may be aligned with the opening of the first shaft when the second shaft extends within the interior bore of the first shaft. A device may include an endoscope, and the first shaft may extend through an opening of the endoscope. The first shaft may include a cap, and the opening of the first shaft may be formed through a sidewall of the cap. The cap may include a shoulder in abutment with a distal end surface of the first shaft. The cap may include an extension portion extending from the shoulder. The connector may be a locking component with a locking element disposed over a locking ring. The connector may include a cryoseal surrounding the second shaft. The cryoseal may include a locking receptacle for receiving the locking element. The connector may be a male-male luer connector. The first shaft may include a cap. The opening of the first shaft may be formed through a sidewall of the cap. The connector may be one of: a male-male luer, or a locking component. The opening of the first shaft may be a substantially rectangular shape. The opening of the first shaft may have a substantially crescent-shaped side profile. The opening provided through the sidewall of the distal end of the first shaft may have an angle configured to direct cryogen spray in a predetermined direction. The opening of the first shaft may include multiple openings.

In the above approach, and various other approaches according to the present disclosure, the inner shaft of the cryosurgery system may extend within an interior bore of the outer shaft. An opening may be provided through the inner shaft for delivery of the cryogen to the opening of the outer shaft. The opening of the inner shaft may be aligned with the opening of the outer shaft when the inner shaft extends within the interior bore of the outer shaft. A system may include an endoscope, and the outer shaft may extend through an opening of the endoscope. The outer shaft may include a cap. The opening of the outer shaft may be formed through a sidewall of the cap. The connector may be one of: a male-male luer, or a locking component. The locking component may include a locking element disposed over a locking ring. A cryoseal may surround the inner shaft. The cryoseal may include a locking receptacle for receiving the locking element. The opening of the outer shaft may be a substantially rectangular shape. The opening of the outer shaft may have a substantially crescent-shaped side profile. The opening provided through the sidewall of the distal end of the outer shaft may have an angle configured to direct cryogen spray in a predetermined direction. The opening of the outer shaft may include multiple openings.

In the above approach, and various other approaches according to the present disclosure, the connector of the catheter may include a stationary component affixed to the outer shaft. The rotary component may be rotatably coupled with the stationary component. The inner shaft may extend within an interior bore of the outer shaft. The catheter may include an opening provided through the inner shaft for delivery of the cryogen to the opening of the outer shaft. The opening of the inner shaft may be aligned with the opening of the outer shaft when the inner shaft extends within the interior bore of the outer shaft. The outer shaft may include a cap. The opening of the outer shaft may be formed through a sidewall of the cap. The cap may include a shoulder in abutment with a distal end surface of the outer shaft. The cap may include an extension portion extending from the shoulder. The connector may be one of: a male-male luer, or a locking component. The locking component may include a locking element disposed over a locking ring. A cryoseal may surround the inner shaft. The cryoseal may include a locking receptacle for receiving the locking element. The opening of the outer shaft may be a substantially rectangular shape. The opening of the outer shaft may have a substantially crescent-shaped side profile. The opening provided through the sidewall of the distal end of the outer shaft may have an angle configured to direct cryogen spray in a predetermined direction. The opening of the outer shaft may include multiple openings.

### BRIEF DESCRIPTION OF THE DRAWINGS

The accompanying drawings illustrate exemplary approaches of the disclosed rotational cryogen delivery device so far devised for the practical application of the principles thereof, and in which:

FIG. 1 is a perspective view of a cryosurgery system according to embodiments of the disclosure;

FIG. 2 is a perspective view of a cryosurgery system according to embodiments of the disclosure;

FIG. 3 is a perspective view of a rotational cryogen delivery device according to embodiments of the disclosure;

FIG. 4A is a side perspective view of an outer shaft of the rotational cryogen delivery device of FIG. 3 according to embodiments of the disclosure;

FIG. 4B is a side cross-sectional view of the outer shaft of the rotational cryogen delivery device of FIG. 3 according to embodiments of the disclosure;

FIG. 4C is a top perspective view of a cap of the outer shaft of the rotational cryogen delivery device of FIG. 3 according to embodiments of the disclosure;

FIG. 5 is a side cutaway view of first and inner shafts of the rotational cryogen delivery device of FIG. 3 according to embodiments of the disclosure;

FIG. 6 is a side perspective view of a connector of the rotational delivery device of FIG. 3 according to embodiments of the disclosure; and

FIG. 7 is a perspective view of a rotational cryogen delivery device according to embodiments of the disclosure.

### DESCRIPTION OF EMBODIMENTS

The present disclosure will now proceed with reference to the accompanying drawings, in which various approaches are shown. It will be appreciated, however, that the disclosed rotational cryogen delivery device may be embodied in many different forms and should not be construed as limited to the approaches set forth herein. Rather, these approaches are provided so that this disclosure will be thorough and complete, and will fully convey the scope of the disclosure to those skilled in the art. In the drawings, like numbers refer to like elements throughout.

As used herein, an element or operation recited in the singular and proceeded with the word “a” or “an” should be understood as not excluding plural elements or operations, unless such exclusion is explicitly recited. Furthermore, references to “one approach” of the present disclosure are not intended to be interpreted as excluding the existence of additional approaches that also incorporate the recited features.

Furthermore, spatially relative terms, such as “beneath,” “below,” “lower,” “central,” “above,” “upper,” and the like, may be used herein for ease of describing one element's relationship to another element(s) as illustrated in the figures. It will be understood that the spatially relative terms may encompass different orientations of the device in use or operation in addition to the orientation depicted in the figures.

As stated above, approaches herein provide a cryospray device including an opening through only a portion of a sidewall thereof, and a connector assembly which allows rotation of the opening for more controlled release of the cryogen from a catheter of the device. In one approach, a connector couples a first shaft (e.g., an outer shaft) to a second shaft (e.g., an inner shaft), the connector including a rotary component connected to a proximal end of the first shaft to allow rotation of the first shaft relative to the second shaft. The opening extends only partially along a circumference of the first shaft to provide a more controlled release of the cryogen from the catheter.

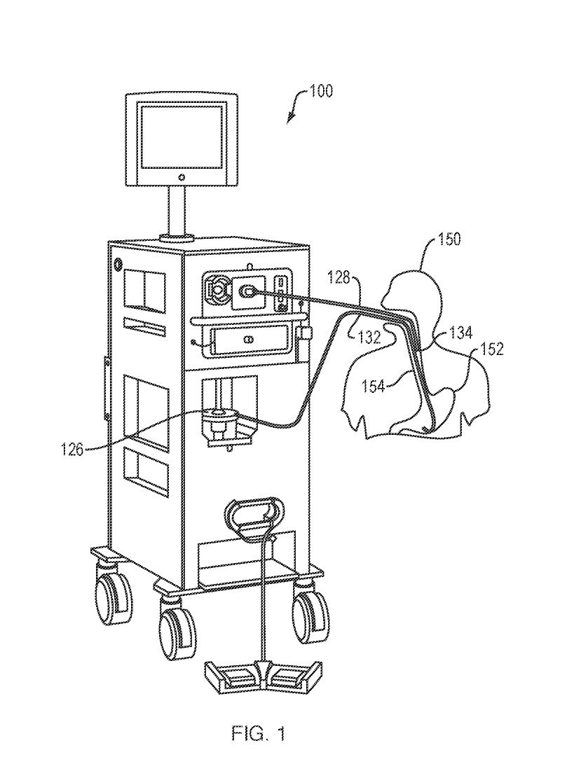

Referring now to FIG. 1, a simplified perspective view of an exemplary cryosurgery system 100 (hereinafter “system”) in which embodiments of the present disclosure may be implemented is illustrated. The system 100 includes a pressurized cryogen storage tank 126 to store cryogen under pressure. In the following description, the cryogen stored in tank 126 is liquid nitrogen, although cryogen may be other materials as described in detail below. The pressure for the liquefied gas in the tank may range from 5 psi to 90 psi in some embodiments. According to one embodiment, pressure in the tank during storage is 40 psi or less, and pressure in the tank during operation is 35 psi or less. According to another embodiment, pressure in the tank during storage is 35 psi or less and pressuring during operation is 25 psi or less. According to yet another embodiment, pressure during operation at normal nitrogen flow is 22±2 psi, and pressure during operation at low nitrogen flow is 14±2 psi. As such, when the pressure in the tank during operation is set to 22 psi, the flow rate/cooling capacity of the nitrogen is 25 W. Alternatively, when the pressure in the tank during operation is set to 14 psi, the flow rate/cooling capacity of the nitrogen is 12.5 W. In an alternate embodiment, the cryogen pressure may be controlled all the way to 45 PSI and to deliver through smaller lumen catheters and additional feature sets. In such alternate embodiments the pressure in the tank during storage may be 55 psi or less. In the context of the output pressure of cryospray from the distal end of the catheter, the term low pressure means 2 psi to 20 psi.

In the embodiment illustrated in FIG. 1, a conventional therapeutic endoscope 134 is used to deliver the nitrogen gas to target tissue within the patient. Endoscope 134 may be of any size, although a smaller diagnostic endoscope is preferably used from the standpoint of patient comfort. In certain embodiments, a specially designed endoscope having a camera integrated therein may also be used. As is known, an image received at the lens on the distal end of the camera integrated into endoscope 134 may be transferred via fiber optics to a monitoring camera which sends video signals via a cable to the a conventional monitor or microscope, where the procedure can be visualized. By virtue of this visualization, the surgeon is able to perform the cryosurgery at treatment site 154 within a patient 150.

As the liquid nitrogen travels from tank 126 to the proximal end of cryogen delivery catheter 128, the liquid is warmed and starts to boil, resulting in cool gas emerging from the distal end or tip of catheter 128. The amount of boiling in catheter 128 depends on the mass and thermal capacity of catheter 128. Since catheter 128 is of small diameter and mass, the amount of boiling is not great. (The catheter may preferably be of size seven French). When the liquid nitrogen undergoes phase change from liquid to gaseous nitrogen, additional pressure is created throughout the length of catheter 128. This is especially true at the solenoid/catheter junction, where the diameter of the supply tube to the lumen of catheter 128 may decrease from approximately 0.25 inches to approximately 0.070 inches, respectively. But the catheter range diameter of its lumen may be between 0.030 to 0.125 inches. In an alternate embodiment the gas boiling inside the catheter may be reduced even greater by the use of insulating materials such as polytetrafluoroethylene (PTFE), fluorinated ethylene propylene (FEP), Pebax® and others to help reduce its temperature coefficient. (Pebax® is a registered trademark in the United States of Arkema France Corporation). The addition of PTFE is especially desirable if done in the inner lumen because its lower coefficient of friction aids in laminar flow of the fluid and thus reducing turbulence and entropy. This reduces gas expansion and allows for good fluid velocity.

When the liquid nitrogen reaches the distal end of catheter 128 it is sprayed out of cryogen delivery catheter 128 onto the target tissue, as will be described in further detail below. It should be appreciated that certain embodiments the cryosurgery system may be able to sufficiently freeze the target tissue without actual liquid nitrogen being sprayed from catheter 128. In particular, a spray of liquid may not be needed if cold nitrogen gas is capable of freezing the target tissue.

Freezing of the target tissue may be visually apparent to the physician by the acquisition of a white color, referred to as cryofrost, within the target tissue. The white color, resulting from surface frost, indicates the onset of mucosal or other tissue freezing sufficient to initiate destruction of the diseased or abnormal tissue. The operator may use the system timer to freeze for a specified duration once initial cryofrost is achieved in order to control the depth of injury. In one embodiment, the composition of catheter 128 or the degree of insulating capacity thereof will be selected so as to allow the freezing of the tissue to be slow enough to allow the physician to observe the degree of freezing and to stop the spray as soon as the surface achieves the desired whiteness of color. The operator may monitor the target tissue to determine when cryofrost has occurred via the camera integrated into endoscope 134. The operator manipulates cryogen catheter 128 to freeze the target tissue. Once the operation is complete, cryodecompression tube 132, catheter 128, and endoscope 134 are withdrawn from the patient 150.

Although not limited herein, catheter length may be anywhere from 10 inches to 100 inches, while inside diameter of the catheter may be anywhere from 0.8 mm to 5 mm, preferably from 1 mm to 4 mm. The tank size may be anywhere from 5 L to 100 L; its diameter may range from 4 inches to 36 inches. The vent orifice of the manifold may be 0.01 inches to 0.1 inches.

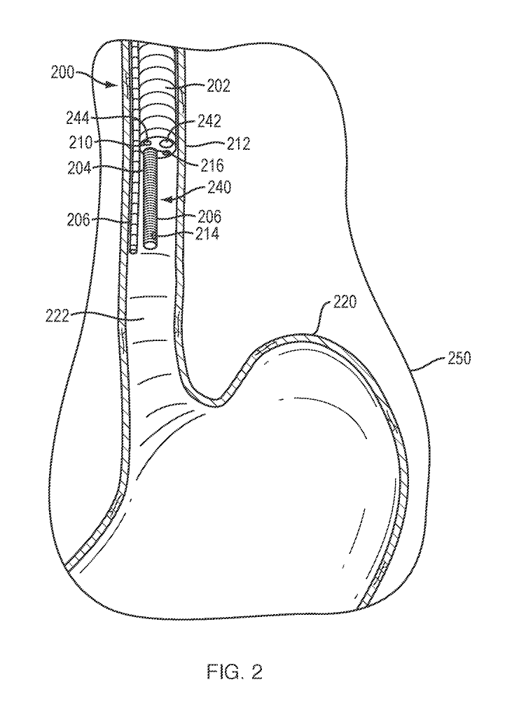

FIG. 2 is a perspective view of a portion of a cryosurgery system 200 having a rotational cryogen delivery device 240. Cryosurgery system 200 comprises an endoscope 202 having lumens 210, 212 and 216 therein. As shown, the endoscope 202 may be positioned in the esophagus 222 of patient the 250 in one non-limiting embodiment. The lumen 212, disposed in the endoscope 202, is configured to receive an endoscope camera 242. Lumen 210 may be configured to receive a light 244 for illumination of the treatment site. Lumen 216 may be configured to receive rotational cryogen delivery device 240. The rotational cryogen delivery device 240 may include a rotation-capable shaft, catheter tip 206, and one or more holes 214, as will be described in greater detail below. After insertion of the cryogen delivery device 240 into the patient, cryogen is provided to the catheter of the cryogen delivery device 240 from a cryogen source (e.g., tank 126 in FIG. 1). The tip 206 causes the cryogen to be sprayed on the target tissue via hole 214. In some embodiments, a dual lumen (for both passive and active venting) cryodecompression tube 208 may be provided to evacuate the treatment area of undesirable gases, particles, fluids etc.

In some embodiments, the controlled pressure and pulsing, coupled with careful control of catheter diameter, length and material composition, helps further deliver controlled flow of volume over time that is consistent with the cryogenic property of the fluid being delivered. Dual phase fluid flow is achieved out of the catheter distal tip and maintained constantly via the equilibrium that the system achieves after pre-cool and after the catheter achieves a cold temperature. The range of dual phase fluid cryogen delivery out of a cryogen catheter with this system can range from 5 LPM to 50 LPM (once it all expands into gas).

In other embodiments, the catheter may be fitted with a temperature sensing probe (not shown) attached to the distal end of the catheter. This may be achieved by laying at least two wires longitudinally or in a coil pattern prior to the outer layer of polymer laminated onto the catheter outer layer. If the wires are thermocouple wires, then the wires can be terminated into a thermocouple. Alternatively, a cryogenic thermistor can be attached to the distal end of the catheter. Such thermistor can then be encapsulated via conductive epoxy and a polymeric sleeve. Then the thermistor can be used to monitor both the temperature at the end of the catheter tip as well as the treatment area for both freezing and thawing temperature monitoring.

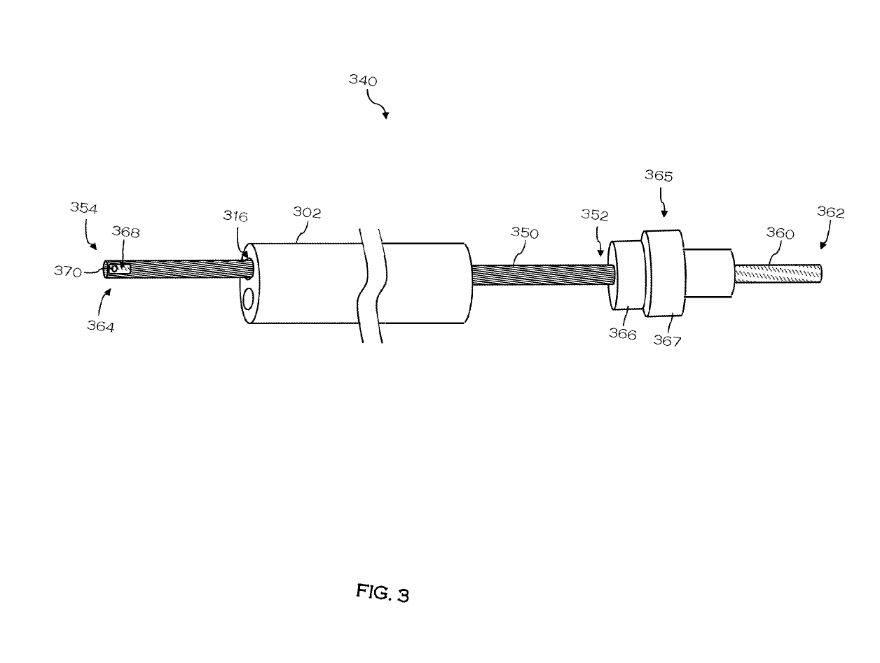

Turning now to FIG. 3, a rotational cryogen delivery device 340 (hereinafter “device”) according to embodiments of the disclosure will be described in greater detail. As shown, the device 340 includes an outer shaft 350 having a proximal end 352 and a distal end 354, wherein the outer shaft 350 extends through the lumen 316 of the endoscope 302. The device 340 further includes an inner shaft 360 having a proximal end 362 and a distal end 364. As shown, the distal end 364 of the inner shaft 360 is adjacent the distal end 354 of the outer shaft 350, as the inner shaft 360 is disposed within the outer shaft 350, as will be described in further detail below.

The outer shaft 350 and the inner shaft 360 are coupled together by a connector 365, thus forming a catheter. The catheter is designed to transport liquid nitrogen (or other cryogen) from the console to the patient treatment site. According to one embodiment, the catheter may contain (1) a bayonet and hub (not shown), (2) a layered polyimide and stainless steel braided shaft to minimize kinking and breaking, (3) insulation to protect the user from cold, (4) a strain relief to help prevent kinking when torqued by users and (5) an atraumatic tip at its distal end to prevent damage to tissue. The laminated construction and braided material provides additional strength and flexibility, allowing the physician to flexibly move the catheter during a treatment procedure, if needed.

According to some embodiments, the catheter formed by the outer shaft 350 and the inner shaft 360 may be constructed from layers of flexible polyimide, surrounded by a stainless steel braid, which is in turn coated with an outer layer of Pebax®. It was discovered that that extrusion of Pebax® over the stainless steel braid allows the Pebax® to wick through the pitch of the steel braid, helping to prevent kinking, breaking, or delamination during retroflex of the catheter. The Pebax® also provides a desirable balance between hardness, which is beneficial for smooth sliding of the catheter and general toughness, and softness, and which provides some degree of tackiness, thus allowing the user to feel the movement of the catheter in the scope. In some embodiments, the pitch of the stainless steel braid is configured to be fine enough to afford the required strength, but not thick enough to allow the Pebax® to wick through.

According to yet another embodiment of the disclosure, the cryospray catheter formed by the outer shaft 350 and the inner shaft 360 may include an all-polymeric shaft construction for the catheter length, and an inner diameter that can be optimized for a specific flow/volume of cryogen within a specific time of spray. Furthermore, the ability of the catheter to deliver cooling can be influenced by the thermal conductivity of the catheter materials and/or construction. Thermally conductive materials can be incorporated into the design to improve the rate of cooling of the catheter materials to help maintain the liquid phase of the flow through such catheter. Certain metals and/or ceramics and/or nano-particles and structures can be incorporated into the polymeric material to increase the heat capacity of the compound(s) from which the catheter is made. One example is the addition of boron nitride into the catheter material. Similarly, support structures in the catheter tube such as braid, coils, and/or longitudinal support members can be incorporated and/or maximized to improve the rate of cooling of the catheter.

As further shown in FIG. 3, in one embodiment, the connector 365 includes a rotary component 366 and a stationary component 367, wherein the rotary component 366 is affixed to the proximal end 352 of the outer shaft 350 and the stationary component 367 is affixed to the inner shaft 360. As arranged, the rotary component 366 is rotatably coupled to the stationary component 367 thereby allowing the outer shaft 350 to rotate relative to the inner shaft 360, as will be described in greater detail below.

An opening 368 is provided through a sidewall of the distal end 354 of the outer shaft 350 for delivery of a cryogen therefrom during use. In some embodiments, one or more openings 370 are provided through the distal end 364 of the inner shaft 360 for delivery of the cryogen to the opening 368 of the outer shaft 350. As demonstrated, the opening 370 of the inner shaft 360 may be aligned with the opening 368 of the outer shaft 350 when the inner shaft 360 is provided within an interior bore of the outer shaft 350. In other embodiments, as will be described in greater detail below, the opening 370 of the inner shaft 360 may be located at one or more different positions along the inner shaft 360 so long as the cryogen emitted therefrom may be directed to the opening 368 of the outer shaft 350.

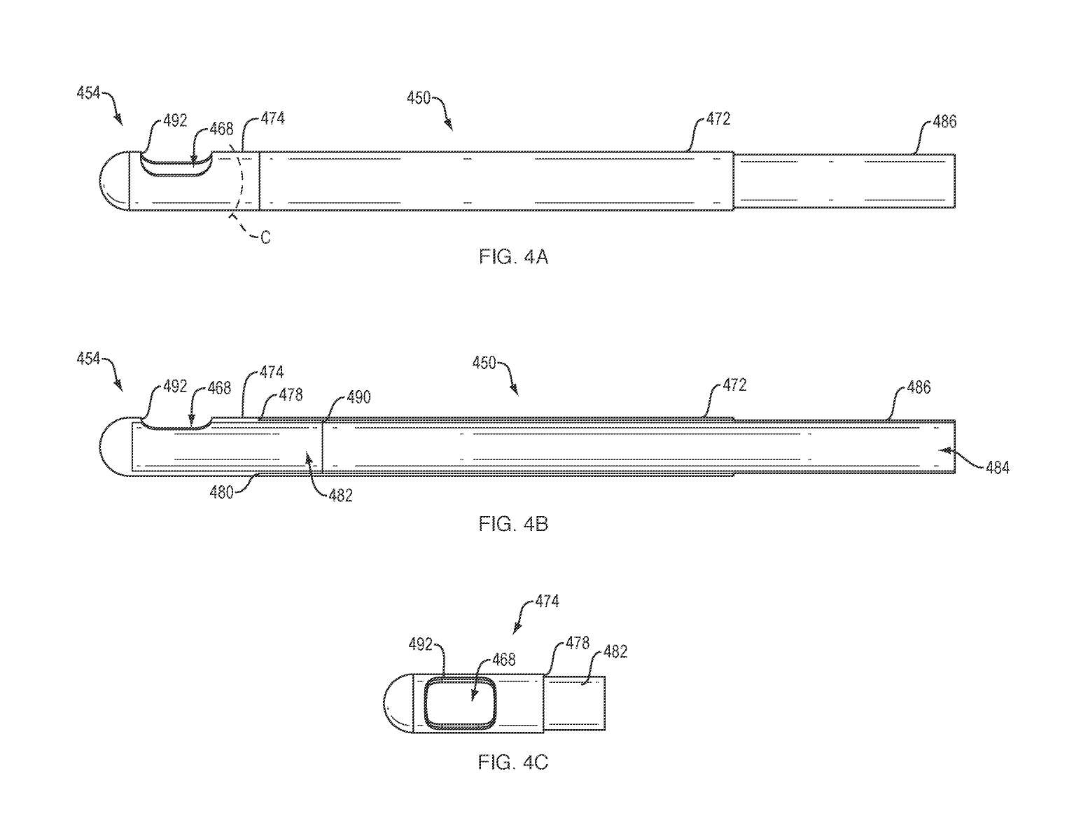

Turning now to FIGS. 4A-C, the outer shaft 450 will be described in greater detail. As shown, the outer shaft 450 may include a cover 472 at the distal end 454 of the outer shaft 450. As further shown, the outer shaft 450 includes a cap 474 coupled to and extending from the cover 472. As best shown in FIGS. 4B-C, the cap 474 includes a shoulder 478 in abutment with a distal end surface 480 of the cover 472, and an extension portion 482 extending from the shoulder 478, away from the distal end 454 of the outer shaft 450. In one embodiment, during assembly, the cap 474 is inserted into an interior 484 of a tube 486 defining a portion of the outer shaft 450. The shoulder 478 of the cap 474 is brought into contact with the distal end surface 480 of the cover 472, and the extension portion 482 is brought into contact with a distal end face 490 of the tube 486. It will be appreciated, however, that this configuration is non-limiting, as other approaches for coupling the cap 474 and the tube 486 are possible within the scope of the present disclosure.

As further shown, the opening 468 of the outer shaft 450 is formed through a sidewall 492 of the cap 474. In exemplary embodiments, the opening 468 is formed only partially along an outer circumference ‘C’ of the outer shaft 450. The opening 468 may be generally rectangular in shape, providing a crescent-shaped side profile. Furthermore, the sidewall 492 of the opening 468 can also be made at a desired angle to direct spray in various directions. It will be appreciated that the opening 468 may be different shapes or include multiple openings in other embodiments.

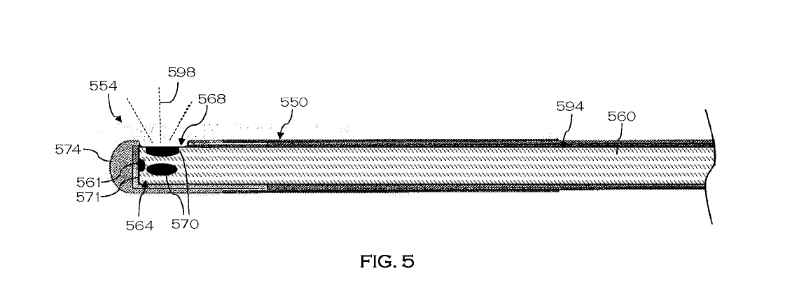

As shown in more detail in FIG. 5, the opening 568 may be provided through the distal end 554 of the outer shaft 550. The inner shaft 560 extends within an interior bore 594 of the outer shaft 550 so that the openings 570 of the inner shaft 560 are generally aligned with the opening 568 through the cap 574 of the outer shaft 550. This arrangement allows delivery of a cryogen 598 through the inner shaft 560 for emission through the opening 568 of the outer shaft 550, e.g., as a side spray. That is, the outer shaft 550 can be positioned and rotated by a user such that the spray of cryogen 598 is output along only a portion of the circumference of the outer shaft 550. The cap 574 thus prevents 360 degree spray of the cryogen 598, thereby better enabling more targeted treatment of only those intended areas, while minimizing the incidental treatment of healthy tissue.

As shown, the openings 570 of the inner shaft 560 are positioned at the distal end 564 thereof. However, the number and positioning of the openings 570 is not limited as such. For example, in other embodiments, the radial spray direction of the openings 570 is supplemented or replaced by an opening 561 through a distal most end surface 571 of the inner shaft 560. The openings 570 may have dimensions that are between 0.005″ to 0.050″ in diameter in some embodiments. The construction of openings 570 may be achieved via drilling of the different hole sizes, fusing or adhering a preformed and predrilled tip.

In the case that the cryogen sprays out of the opening 561 at the distal most end surface 571 of the inner shaft 560, the liquid nitrogen may be broken down into small droplets via a diffuser or filter (not shown) to allow for an even spray pattern and avoid cold spots of spray pattern. The diffuser may be constructed of filter paper, a grating patterned polymer, a metal or plastic mesh basket or laser cutting methods on the shaft itself to pattern it with very small holes. In such embodiment, the inner shaft 560 may end in a cap that contains small longitudinal cuts that provide for controlled spray to exit as it initially hits a bounce plate (not shown). The bounce plate may be of a conical shape and helps distribute the spray evenly all around the distal end 564 of the inner shaft 560.

In other embodiments, the inner shaft 560 may include openings of different sizes and/or at different distance positions to allow for gradual spray across a specific distance of the inner shaft 560. For example, various hole patterns may consist of varying numbers of rows, varying hole sizes, number of holes per row, number of slits instead of rows, separation between holes, spiral hole patterns around the circumference, and variable hole patterns to compensate flow along the length of shaft. Slits can either be vertical or horizontal with respect to the shaft length. Individual hole sizes can vary from Outer Diameter to Inner Diameter. The holes can also be made at an angle within the wall thickness of the tube to direct spray in various directions.

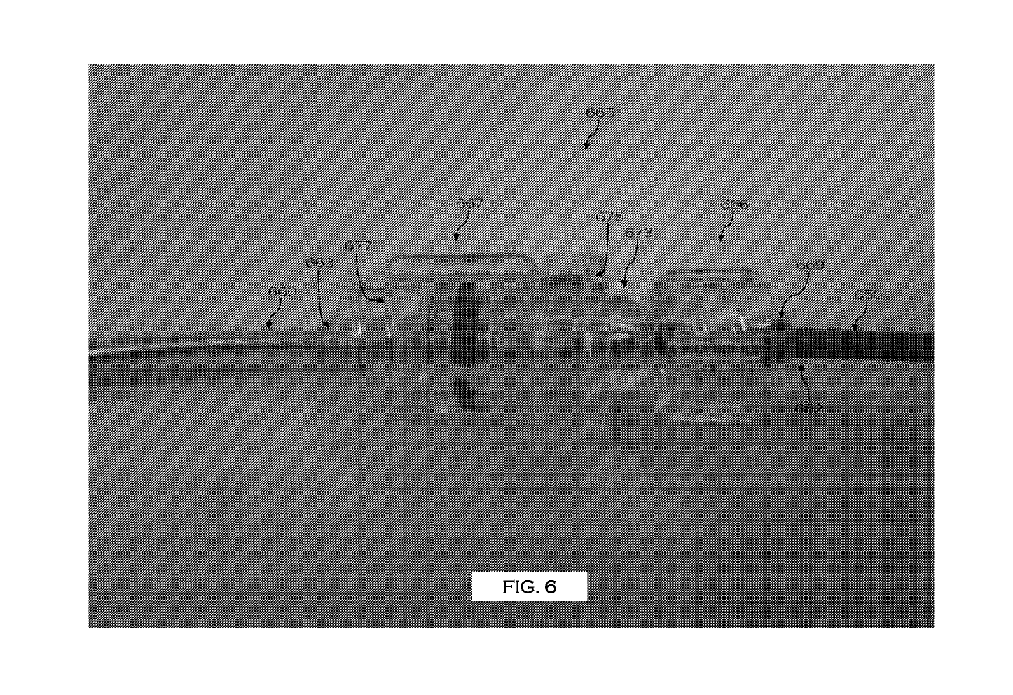

Turning now to FIG. 6, a connector 665 for use with a rotational cryogen delivery device will be described in greater detail. As shown, the connector 665 includes a rotary component 666 and a stationary component 667, wherein the rotary component 666 is affixed to the proximal end 652 of the outer shaft 650, and the stationary component 667 is affixed to the inner shaft 660. As arranged, the rotary component 666 is rotatably coupled to the stationary component 667 to allow rotation of the outer shaft 650 relative to the inner shaft 660.

More specifically, the connector 665 may be a male-male luer connector, wherein a first retaining member 669 of the rotary component 666 surrounds and engages the outer shaft 650 so as to prevent rotation of the outer shaft 650 relative to the rotary component 666. Coupling of the outer shaft 650 to the rotary component 666 may be accomplished via press fit or by one or more mechanical attachment components. A second retaining member 663 of the stationary component 667 surrounds and engages the inner shaft 660 so as to prevent rotation of the inner shaft 660 relative to the stationary component 667. Coupling of the inner shaft 660 to the stationary component 667 may be accomplished via press fit or by one or more mechanical attachment components.

In some embodiments, an interior column 673 of the rotary component 666 extends into a central cavity 675 of the stationary component 667, and may include exterior threading along a surface of the interior column 673 for engagement with corresponding threading 677 provided along a surface of the central cavity 675. The threaded engagement between the interior column 673 and the stationary component 667 allows the rotary component 666 to be rotated within and move axially with respect to the stationary component 667. Beneficially, the outer shaft 650 can freely rotate 360 degrees, thus allowing for better controlled spray of the cryogen.

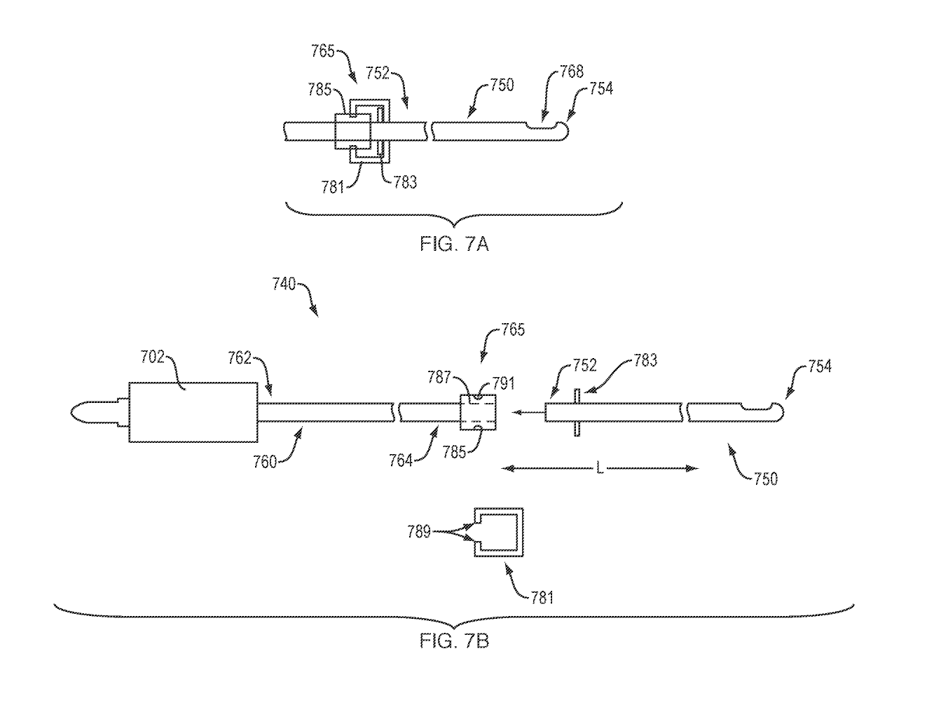

Turning now to FIGS. 7A-B, a rotational cryogen delivery device 740 (hereinafter “device”) according to embodiments of the disclosure will be described in greater detail. As shown, the device 740 includes an outer shaft 750 having a proximal end 752 and a distal end 754, wherein the outer shaft 750 may extend through an endoscope 702. The device 740 further includes an inner shaft 760 having a proximal end 762 and a distal end 764. The outer shaft 750 and the inner shaft 760 are coupled together by a connector 765, thus forming a catheter for delivery of a cryogen spray therefrom. In this embodiment, the inner shaft 760 does not extend within an interior bore of the outer shaft 750. Instead, the inner shaft 760 terminates proximate the connector 765.

As shown, an opening 768 is provided through a sidewall of the distal end 754 of the outer shaft 750 for delivery of a cryogen during use. In exemplary embodiments, the opening 768 is formed only partially along an outer circumference of the outer shaft 750. The opening 768 may include a crescent-shaped side profile so as to provide only a side spray of cryogen during use. By limiting the size and position of the opening 768, cryogen spray is better directed towards only an intended treatment location.

As shown, the connector 765 includes a locking component and a seal, wherein the locking component includes a locking element 781 disposed over a locking ring 783, and the seal includes a cryoseal 785 surrounding the inner shaft. As shown, the locking element 781 is configured to engage the cryoseal 785, and includes a cavity for receiving the locking ring 783 therein. During use, the locking ring 783 abuts an interior wall of the locking element to limit movement along a lengthwise axis I′, while still allowing rotation of the outer shaft 750 relative to the inner shaft 760. As shown, the outer shaft 750 may be inserted into a bore 787 of the cryoseal 785, and end sections 789 of the locking element 781 may be secured within a set of locking receptacles 791 of the cryoseal 785. The locking ring 783 is thus encased by the locking element 781, limiting lateral movement yet still enabling rotation of the outer shaft 750.

As will be appreciated, embodiments of the present disclosure described herein advantageously minimize the impact to healthy tissue caused by radial spray, which typically offers 360 degree impact, by providing a catheter including an opening through only a portion of a sidewall thereof. A connector assembly allows rotation of the opening for more controlled release of the cryogen from the catheter.

While the present disclosure has been described with reference to certain approaches, numerous modifications, alterations and changes to the described approaches are possible without departing from the sphere and scope of the present disclosure, as defined in the appended claims. Accordingly, it is intended that the present disclosure not be limited to the described approaches, but that it has the full scope defined by the language of the following claims, and equivalents thereof. While the disclosure has been described with reference to certain approaches, numerous modifications, alterations and changes to the described approaches are possible without departing from the spirit and scope of the disclosure, as defined in the appended claims. Accordingly, it is intended that the present disclosure not be limited to the described approaches, but that it has the full scope defined by the language of the following claims, and equivalents thereof.

What is claimed is:

1. A rotational cryogen delivery device comprising:
a first shaft having a proximal end and a distal end;
a second shaft having a proximal end and a distal end;
a connector coupling the first shaft to the second shaft, the connector including a rotary component connected to the proximal end of the first shaft to allow rotation of the first shaft relative to the second shaft; and
an opening provided through a sidewall of the distal end of the first shaft for delivery of a cryogen, wherein the opening extends only partially along a circumference of the first shaft.

2. The rotational cryogen delivery device of claim 1, the connector further including a stationary component affixed to the second shaft.

3. The rotational cryogen delivery device of claim 2, wherein the rotary component is rotatably coupled with the stationary component.

4. The rotational cryogen delivery device of claim 1, wherein the second shaft extends within an interior bore of the first shaft.

5. The rotational cryogen delivery device of claim 4, further comprising an opening provided through the second shaft for delivery of the cryogen to the opening of the first shaft.

6. The rotational cryogen delivery device of claim 5, wherein the opening of the second shaft is aligned with the opening of the first shaft when the second shaft extends within the interior bore of the first shaft.

7. The rotational cryogen delivery device of claim 1, the first shaft comprising a cap, wherein the opening of the first shaft is formed through a sidewall of the cap.

8. A catheter comprising:
an outer shaft having a proximal end and a distal end;
an inner shaft having a proximal end and a distal end;
a connector coupling the outer shaft to the inner shaft, the connector including a rotary component connected to the proximal end of the outer shaft to allow rotation of the outer shaft relative to the inner shaft; and
an opening provided through a sidewall of the distal end of the outer shaft for delivery of a cryogen, wherein the opening extends only partially along a circumference of the outer shaft.

9. The catheter of claim 8, the connector further including a stationary component affixed to the outer shaft.

10. The catheter of claim 9, wherein the rotary component is rotatably coupled with the stationary component.

11. The catheter of claim 8, wherein the inner shaft extends within an interior bore of the outer shaft.

12. The catheter of claim 11, further comprising an opening provided through the inner shaft for delivery of the cryogen to the opening of the outer shaft.

13. The catheter of claim 12, wherein the opening of the inner shaft is aligned with the opening of the outer shaft when the inner shaft extends within the interior bore of the outer shaft.

14. The catheter of claim 8, the outer shaft comprising a cap, wherein the opening of the outer shaft is formed through a sidewall of the cap.

15. The catheter of claim 8, wherein the connector is one of: a male-male luer, or a locking component, wherein the locking component comprises:
a locking element disposed over a locking ring; and
a cryoseal surrounding the inner shaft, wherein the cryoseal includes a locking receptacle for receiving the locking element.

16. A cryosurgery system comprising:
an outer shaft having a proximal end and a distal end;
an inner shaft having a proximal end and a distal end;
a connector coupling the outer shaft to the inner shaft, the connector including:
a rotary component affixed to the proximal end of the outer shaft to allow rotation of the outer shaft relative to the inner shaft; and
a stationary component affixed to the inner shaft, the rotary component rotatably coupled to the stationary component; and
an opening provided through a sidewall of the distal end of the outer shaft for delivery of a cryogen, wherein the opening extends only partially along a circumference of the outer shaft.

17. The cryosurgery system of claim 16, wherein the inner shaft extends within an interior bore of the outer shaft.

18. The cryosurgery system of claim 16, further comprising an opening provided through the inner shaft for delivery of the cryogen to the opening of the outer shaft.

19. The cryosurgery system of claim 16, the outer shaft comprising a cap, wherein the opening of the outer shaft is formed through a sidewall of the cap.

20. The cryosurgery system of claim 16, wherein the connector is one of: a male-male luer, or a locking component, the locking component comprising:
a locking element disposed over a locking ring; and
a cryoseal surrounding the inner shaft, wherein the cryoseal includes a locking receptacle for receiving the locking element.
